(19) United States
(12) Patent Application Publication
(10) Pub. No.: US 2011/0086000 A1
(43) Pub. Date: Apr. 14, 2011

(54) NOVEL SKIN PEEL COMPOSITION IN MASQUE FORM

(75) Inventors: Sue Feng, Edison, NJ (US); Gina Rousseau, Westfield, NJ (US)

(73) Assignee: L'ORÈAL S.A., Paris (FR)

(21) Appl. No.: 12/895,125

(22) Filed: Sep. 30, 2010

Related U.S. Application Data
(60) Provisional application No. 61/250,301, filed on Oct. 9, 2009.

(51) Int. Cl. A61K 8/81; A61Q 19/08

(52) U.S. Cl. 424/78.03

(57) ABSTRACT

The present invention is directed to a method of reducing the appearance of skin changes associated with intrinsic and/or extrinsic aging, involving the steps of: (a) providing a first composition containing: (i) from about 20 to about 50% by weight of at least one hydroxy acid; (ii) optionally, from about 1 to about 15% by weight of at least one weak organic acid; and (iii) remainder, to 100%, water; (b) providing a second composition containing: (iv) from about 20 to about 50% by weight of L-ascorbic acid powder; (v) optionally, from about 1 to about 10% by weight of an extract of emblica; (vi) from about 10 to about 60% by weight of a water-soluble film-forming polymer; and (vii) remainder, to 100%, of a water-absorbing thickening agent; (c) mixing (a) and (b) to form a finished composition; (d) applying the finished composition onto the skin, just after mixing, in order to form a masque on the skin; and (e) removing the masque from the skin after a predetermined period of time.

### REFERENCE TO PRIOR APPLICATION

This application claims the benefit of priority under 35 U.S.C. §119(e) from U.S. provisional application Ser. No. 61/250,301, filed Oct.9, 2009, incorporated herein by reference in its entirety.

### FIELD OF THE INVENTION

The present invention is directed to a novel skin peel composition which, when applied onto the skin, forms an efficacious masque. More particularly, the invention relates to a composition containing high levels of both a hydroxy acid and ascorbic acid in a carrier which, after being applied onto skin, forms a masque.

### BACKGROUND OF THE INVENTION

Skin renewal can be stimulated by removal of the outer keratinous layer of the skin system. Such removal can be effected mechanically, for example, by rubbing with an abrasive composition, or chemically. Chemical exfoliation and peeling are effected by agents that interact with the complex structure of the skin, removing the outer layer to expose the underlying layers. Skin-renewal stimulating compositions and agents can provide anti-aging benefits, for example, a reduction of keratoses, freckles, wrinkles, fine lines and epidermal and dermal atrophy. The newly exposed skin is smoother and appears younger.

In recent years the use of cosmetic and pharmacological formulations containing skin-renewal simulating acids, such as alpha hydroxy acids (“AHAs”) or retinoic acid, to obtain antiaging and dermatologically therapeutic benefits has become widespread. Scientific and clinical reports, as well as much subjective evidence, have shown that substantial improvements in skin appearance and condition can be obtained by means of skin-renewal stimulating acids, which are believed to be attributable to increased rates of skin cell renewal, and the removal of outer layers of dead cells.

Chemical peeling can be done in varying degrees of depth. A light or superficial peel is generally one which is comparatively superficial in effect, and medium or deep chemical peels are ones in which peeling agents are used to produce a moderate to severe wound to the skin. Medium or deep peels achieve a much more dramatic and visible effect, and do so quickly, in minutes or hours, but usually result in pain and inflammation of the skin.

It is an object of this invention to provide a safe and highly efficacious peel for reducing the appearance of skin changes associated with intrinsic and/or extrinsic aging.

It is another object of this invention to provide a safe and highly efficacious peel for use in the treatment of regional hyperpigmentation caused by melanocytic hyperactivity, as well as for localized hyperpigmentation and benign melanocytic proliferations, such as senile pigmentary spots, actinic photosensitization and post-lesion scarring.

It is still another object of this invention to provide a selection of peels beneficial in the treatment and prevention of acne.

### SUMMARY OF THE INVENTION

The present invention is directed to a method of reducing the appearance of skin changes associated with intrinsic and/or extrinsic aging, involving the steps of:
(i) providing a first composition containing:
   (a) from about 20 to about 50% by weight of at least one hydroxy acid;
   (b) optionally, from about 1 to about 15% by weight of at least one weak organic acid; and
   (c) remainder, to 100%, water;
providing a second composition containing:
   (d) from about 20 to about 50% by weight of L-ascorbic acid powder;
   (e) optionally, from about 1 to about 10% by weight of an extract of emblica;
   (f) from about 10 to about 60% by weight of a water-soluble film-forming polymer; and
   (g) remainder, to 100%, of a water-absorbing thickening agent;
(iii) mixing (i) and (ii) to form a finished composition;
(iv) applying the finished composition onto the skin, just after mixing, in order to form a masque on the skin; and
(v) removing the masque from the skin after a predetermined period of time.

The present invention is also directed to a kit for use in reducing the appearance of skin changes associated with intrinsic and/or extrinsic again, the kit comprising a multi-unit receptacle having:
(a) a first unit containing:
(i) from about 20 to about 50% by weight of at least one hydroxy acid;
(ii) optionally, from about 1 to about 15% by weight of at least one weak organic acid; and
(iii) remainder, to 100%, water;
   (b) a second unit containing:
(iv) from about 20 to about 50% by weight of L-ascorbic acid powder;
(v) optionally, from about 1 to about 10% by weight of an extract of emblica;
(vi) from about 10 to about 60% by weight of a water-soluble film-forming polymer; and
(vii) remainder, to 100%, of a water-absorbing thickening agent; and
   (c) optionally, a third unit for mixing the contents of (a) and (b).

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

First Composition

According to one aspect of the present invention, there is provided a first composition containing a mixture of a hydroxyl acid and water.

Suitable hydroxy acids for use in the first composition include hydroxymonocarboxylic acids such as, for example, glycolic acid; lactic acid; glyceric acid, and the like. A particularly preferred hydroxyl acid is glycolic acid.

The hydroxy acid is typically present in the first composition in an amount of from about 20 to about 60% by weight, such as from about 25 to about 50% by weight, and from about 30 to about 45% by weight, all weights being based on the total weight of the composition.

It may also be desirable to employ at least one weak organic acid. Examples of suitable weak organic acids include, but are not limited to, citric acid; lactic acid; acetic acid; formic acid; and oxalic acid. A particularly preferred weak organic acid is citric acid.

The weak organic acid, if employed, will typically be present in an amount ranging from about 1 to about 15% by weight, such as from about 5 to about 12% by weight, and from about 7 to about 10% by weight, all weights based on the total weight of the first composition.

In an effort to accelerate evaporation time for the first composition, it may also be desirable to employ a co-solvent having a high flash-point. An example thereof is an alcohol, such as ethanol. A particularly preferred co-solvent is denatured alcohol. The co-solvent, if employed, is typically present in an amount of from about 1 to about 10% by weight, such as from about 1 to about 7% by weight, and from about 3 to about 5% by weight, all weights being based on the weight of the first composition.

Second Composition

The present invention also employs a second composition, in powder form, the composition containing a combination of L-ascorbic acid, an extract of emblica, at least one water-soluble film forming polymer, and a water-absorbing thickening agent.

The L-ascorbic acid is preferably employed in an amount of from about 20 to about 50% by weight, such as from about 20 to about 40% by weight, and from about 25 to about 30% by weight, all weights being based on the total weight of the second composition.

As used herein, the terms “an extract of Emblica officinalis,” “emblica extract” or “an extract of emblica” mean any extract of the fruit of the Emblica officinalis or Phyllanthus emblica tree. Such extracts may be prepared by any method known to those skilled in the art. In addition, the terms “an extract of Emblica officinalis,” “emblica extract” and “an extract of emblica” mean any compounds or mixtures of compounds that are isolated or purified from crude extracts of the fruit of the tree. Such compounds or mixtures of compounds may be isolated or purified by any method known to those skilled in the art. For example, compositions comprisingemblica extract, as well as its individual chemical components are described in U.S. Pat. No. 6,124,268, the entire contents of which are hereby incorporated by reference.

The extract of emblica is generally employed in an amount of from about 1 to about 10% by weight, such as from about 2 to about 8% by weight, and from about 3 to about 5% by weight, all weights being based on the total weight of the second composition.

Examples of water-soluble film-forming polymers which may be mentioned are: proteins, for instance proteins of plant origin such as wheat proteins and soybean proteins; proteins of animal origin such as keratins, for example keratin hydrolysates and sulfonic keratins; anionic, cationic, amphoteric or nonionic chitin or chitosan polymers; polymers of celluloses such as hydroxyethylcellulose, hydroxypropylcellulose, methylcellulose, ethylhydroxyethylcellulose and carboxymethylcellulose, and quaternized cellulose derivatives; acrylic polymers or copolymers, such as polyacrylates or polymethacrylates; vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol; polymers of natural origin, which are optionally modified, such as: gum arabics, guar gum, xanthan derivatives, karaya gum; alginates and carrageenans; glycoaminoglycans, hyaluronic acid and derivatives thereof; shellac resin, sandarac gum, dammar resins, elemi gums and copal resins; deoxyribonucleic acid; mucopolysaccharides such as hyaluronic acid and chondroitin sulfate, and mixtures thereof.

Particularly preferred water-soluble film-forming polymers are copolymers of vinylpyrrolidone and of vinyl acetate. The polymer is preferably employed in an amount of from about 10 to about 60% by weight, such as from about 20 to about 55% by weight, and from about 30 to about 50% by weight,. all weights being based on the total weight of the composition.

Suitable water-absorbing thickening agents include, for example, corn starch, flour, wheat starch, and the like. A particularly preferred water-absorbing thickening agent is corn starch. The water-absorbing thickening agent is typically employed in an amount of from about 20 to about 60% by weight, such as from about 25 to about 50% by weight, and from about 30 to about 40% by weight, all weights being based on the total weight of the second composition.

Auxiliary Ingredients

The first and/or second compositions of the present invention may also include, if desired, various types of auxiliary ingredients. Examples thereof include preservatives, conditioning agents, and the like.

According to one embodiment of the present invention, there is provided a method of reducing the appearance of skin changes associated with intrinsic and/or extrinsic aging, involving the steps of: (a) providing a first composition containing: (i) from about 20 to about 50% by weight of at least one hydroxy acid; (ii) optionally, from about 1 to about 15% by weight of at least one weak organic acid; and (iii) remainder, to 100%, water; (b) providing a second composition containing: (iv) from about 20 to about 50% by weight of L-ascorbic acid powder; (v) optionally, from about 1 to about 10% by weight of an extract of emblica; (vi) from about 10 to about 60% by weight of a water-soluble film-forming polymer; and (vii) remainder, to 100%, of a water-absorbing thickening agent; (c) mixing (a) and (b) to form a finished composition; (d) applying the finished composition onto the skin, just after mixing, in order to form a masque on the skin; and (e) removing the masque from the skin after a predetermined period of time.

It is imperative that the first and second compositions are mixed just prior to application onto the skin, so as to minimize oxidative degradation of L-ascorbic acid. It is equally important that second composition be in powder form, prior to its being mixed with the first composition, so as to inhibit exposure of the L-ascorbic acid to water/moisture.

Once the first and second composition and mixed, in situ, to form the finished composition, it is then applied onto a target area of the skin for a predetermined period of time. The precise period of time will depend on the particular skin change being addressed, whether hyper pigmentation, acne, wrinkles, or some other appearance of skin change associated with intrinsic and/or extrinsic aging.

According to yet another embodiment of the present invention, there is provided a kit for reducing the appearance of skin changes associated with intrinsic and/or extrinsic aging, the kit comprising a multi-unit receptacle having: (a) a first unit containing: (i) from about 20 to about 50% by weight of at least one hydroxy acid; (ii) optionally, from about 1 to about 15% by weight of at least one weak organic acid; and (iii) remainder, to 100%, water; (b) a second unit containing: (iv) from about 20 to about 50% by weight of L-ascorbic acid powder; (v) optionally, from about 1 to about 10% by weight of an extract of emblica; (vi) from about 10 to about 60% by weight of a water-soluble film-forming polymer; and (vii) remainder, to 100%, of a water-absorbing thickening agent; and (c) optionally, a third unit in which the contents of (a) and (b) can be mixed.

The present invention will be better understood from the examples which follow, all of which are intended for illustrative purposes only and are not meant to unduly limit the scope of the invention in any way.

### EXAMPLES

Example 1 shows a first and second composition used for reducing the appearance of skin hyper pigmentation.

Composition one contains the following:

| Type | Nom INCI US | % |
|---|---|---|
| ACTI-1 | GLYCOLIC ACID | 40.0000 |
| ACTI-1 | CITRIC ACID | 10.0000 |
| SOLV-1 | ALCOHOL DENAT. | 5.0000 |
| SOLV-1 | WATER | 45.0000 |

Composition two contains the following:

| Type | Nom INCI US | % |
|---|---|---|
| ACTI-1 | ALLANTOIN | 0.2000 |
| ACTI-1 | DISODIUM EDTA | 0.1000 |
| BOT-1 | ALOE BARBADENSIS LEAF JUICE | 0.3500 |
| BOT-1 | PHYLLANTHUS EMBLICA FRUIT EXTRACT | 4.0000 |
| C20690792 | SILICA | 2.5000 |
| POLY-1 | VP/VA COPOLYMER | 23.1500 |
| POLY-1 | ZEA MAYS (CORN) STARCH | 40.0000 |
| POLY-1 | CELLULOSE GUM | 1.5000 |
| VITA-1 | PANTHENOL | 1.2000 |
| VITA-1 | ASCORBIC ACID | 27.0000 |

Compositions 1 and 2 were mixed together to form a finished composition. The finished composition was then immediately applied onto a target area of skin in order to reduce the appearance of signs of hyperpigmentation, and left there for approximately 5 minutes. Once applied onto the skin, the finished composition began to form a masque, enabling the actives contained within the masque to perform more efficaciously on the target area. Afterwards, the masque was rinsed from the target area.

What is claimed is:

1. A method of reducing the appearance of skin changes associated with intrinsic and/or extrinsic aging comprising:
(i) providing a first composition containing:
(a) from about 20 to about 50% by weight of at least one hydroxy acid;
(b) optionally, from about 1 to about 15% by weight of at least one weak organic acid; and
(c) remainder, to 100%, water;
(ii) providing a second composition containing:
(d) from about 20 to about 50% by weight of L -ascorbic acid powder;
(e) optionally, from about 1 to about 10% by weight of an extract of emblica;
(f) from about 10 to about 60% by weight of a water-soluble film-forming polymer; and
(g) remainder, to 100%, of a water-absorbing thickening agent;
(iii) mixing (i) and (ii) to form a finished composition;
(iv) applying the finished composition onto the skin, just after mixing, in order to form a masque on the skin; and
(v) removing the masque from the skin after predetermined period of time.

2. The method of claim 1 wherein (a) is glycolic acid.

3. The method of claim 1 wherein (b) is citric acid

4. The method of claim 1 wherein (f) is a copolymer of vinylpyrrolidone and vinylacetate.

5. The method of claim 1 wherein (g) is corn starch.

6. A kit for reducing the appearance of skin changes associated with intrinsic and/or extrinsic aging comprising a multi-unit receptacle containing:
(a) a first unit containing:
(i) from about 20 to about 50% by weight of at least one hydroxy acid;
(ii) optionally, from about 1 to about 15% by weight of at least one weak organic acid; and
(iii) remainder, to 100%, water;
(b) a second unit containing:
(iv) from about 20 to about 50% by weight of L-ascorbic acid powder;
(v) optionally, from about 1 to about 10% by weight of an extract of emblica;
(vi) from about 10 to about 60% by weight of a water-soluble film-forming polymer; and
(vii) remainder, to 100%, of a water-absorbing thickening agent; and
(c) optionally, a third unit for mixing the contents of (a) and (b).

7. The kit of claim 6 wherein (a)(i) is glycolic acid.

8. The kit of claim 6 wherein (a)(ii) is citric acid.

9. The kit of claim 6 wherein (b)(vi) is a copolymer of vinyl pyrrolidone and vinyl acetate.

10. The kit of claim 6 wherein (b)(vii) is corn starch.
